(19) United States
(12) Patent Application Publication
(10) Pub. No.: US 2008/0187935 A1
(43) Pub. Date: Aug. 7, 2008

(54) Homo and Heterodimer Proteins of the Abcg Family, Methods For Detection and Screening Modulators Thereof

(76) Inventors: Judit Cserepes, Budapest (HU); N. Barry Elkind, Toronto (CA); Zsofia Szentpetery, Godollo (HU); Balazs Sarkadi, Budapest (HU); Andras Varadi, Budapest (HU); Izabella Klein, Budapest (HU); Laszlo Homolya, Telki (HU); Laszlo Seres, Budapest (HU); Csilla Ozvegy-Laczka, Budapest (HU)

Correspondence Address: HAHN & VOIGHT PLLC, 1012 14TH STREET, NW, SUITE 620, WASHINGTON, DC, 20005, US

(21) Appl. No.: 11/571,754

(22) PCT Filed: Jul. 8, 2005
(86) PCT No.: PCT/HU05/00074

§ 371 Date: Aug. 23, 2007

(30) Foreign Application Priority Data
Jul. 8, 2004 (HU) P0401380

(51) Int. Cl. G01N 33/53; C12Q 1/34; C07K 16/00; C07K 14/00; C12Q 1/02

(52) U.S. Cl. 435/7.1; 435/18; 435/29; 530/388.1; 530/387.1; 530/402

(57) ABSTRACT

The invention relates to methods for screening selective modulators of half transporter proteins of the ABCG family, more closely of ABCG1 and ABCG4. In particular the invention relates to methods for determining whether a substance is a selective activator, an inhibitor or a substrate of an ABCG1 or ABCG4 homodimer or of an ABCG1/ABCG4 heterodimer protein, methods for detection of ABCG1 protein in a biological sample, methods for modulating the function of said proteins, and methods for detecting the presence of and/or quantitating ABCG1/ABCG4 heterodimer activity in a biological sample. Moreover, the invention relates to isolated ABCG1/ABCG4 heterodimer proteins and antibodies selective for ABCG1 or ABCG4. The closely related human ABC half-transporters, ABCG1 and ABCG4, have been suggested to play an important role in cellular lipid/sterol regulation. ABCG1 and ABCG4 and mutants thereof have been expressed and studied by the present inventors in whole cells as well as isolated membrane preparations. A large number of compounds have been screened in this system. Co-expression of the ABCG1 and ABCG4 half transporters resulted in heterodimers.

The invention relates to methods for screening selective modulators of half transporter proteins of the ABCG family, more closely of ABCG1 and ABCG4. In particular the invention relates to methods for determining whether a substance is a selective activator, an inhibitor or a substrate of an ABCG1 or ABCG4 homodimer or of an ABCG1/ABCG4 heterodimer protein, methods for detection of ABCG1 protein in a biological sample, methods for modulating the function of said proteins, and methods for detecting the presence of and/or quantitating ABCG1/ABCG4 heterodimer activity in a biological sample. Moreover, the invention relates to isolated ABCG1/ABCG4 heterodimer proteins and antibodies selective for ABCG1 or ABCG4.

### BACKGROUND ART

The five members of the human ATP-binding cassette (ABC) G subfamily of transporters (ABCG1, ABCG2, ABCG4, ABCG5 and ABCG8) have a unique domain structure consisting of one single nucleotide binding domain (NBD) located N-terminally of the six pass transmembrane domain (TMD) (for review Klein, I et al., 1999, FIG. 1). These half-transporters have to homo- or heterodimerize in order to form functionally active transporters. ABCG2 is thought to act as homodimer (Özvegy, C et al. 2002, Mitomo, H et al. 2003) while ABCG5 and G8 function as an obligatory heterodimeric complex (Graf, G A et al., 2003).

Human ABCG2 (BCRP/MXR/ABCP) is a well characterized member of the ABCG family. The overexpression of ABCG2 in drug-resistant cell lines and tumors, as well as its demonstrated transport activity for a number of clinically applied antitumor agents, suggests an important role for this protein in cancer multidrug resistance. In addition, ABCG2 is expressed in stem cells, placenta, liver, small intestine, colon, lung, kidney, adrenal and sweat glands, and in the endothelia, suggesting its important role in protection against xenobioties. Homodimerization, the ATP-dependent active transport function and the molecular mechanism of ABCG2, as well as of its mutant and polymorphic variants have been analyzed in several experimental studies in various expression systems (for review see Haimeur, A et al., 2004, Sarkadi B et al., 2004).

It is well documented that ABCG5 and ABCG8 function as heterodimeric active transporters for sitosterols and probably also for cholesterol and cholesterol derivatives. The inherited disease, sitosterolemia is caused by a mutation in either one of these proteins, and the proper plasma membrane localization and function of ABCG5 and ABCG8 is only achieved when they form heterodimers and co-processed by the cellular expression machinery (Graf G A et al., 2003).

There is much less information known as yet about the function, localization, and the mechanism of action of ABCG1 and ABCG4. The ABCG1 (ABC8) gene and its putative gene product were independently recognized by two groups as the Drosophila white gene homologue (Croop J M et al., 1997, Chen H et al., 1996). The human ABCG1 mRNA was found to be expressed primarily in the heart, spleen, brain, liver, lung, skeletal muscle, kidney and placenta (Croop J M et al., 1997, Chen H et al., 1996, Klucken J et al., 2000, Oldfield S et al., 2002). In human macrophages elevated expression of ABCG1 mRNA was identified subsequent to cholesterol loading (Klucken J et al., 2000, Venkateswaran A et al., 2000, Laffitte B et al., 2001), oxidized LDL treatment, or upon the addition of LXR and RXR agonists (Engel T et al., 2001). Thus, a growing body of evidence indicates ABCG1 involvement in lipid/sterol regulation (for review see Schmitz G et al., 2001). According to initial studies in human cells, endogenous ABCG1 was found to localize to both plasma membrane and to internal membranes (Klucken J et al., 2000, Lorkowski S et al., 2001).

Human ABCG4 was identified independently in several laboratories based on its homology and close similarity to ABCG1, and its cDNA was cloned from testes libraries (Oldfield S et al. 2002, Annilo T et al., 2001).

Nucleotide sequences encoding human ABCG4 protein are also disclosed in US20030166885A1 and WO02070691A2 patent applications (Chen H and Le Bihan, S). Amino acid sequence and the corresponding cDNA of a closely related protein denominated as 52948 is disclosed in US2003/0166885 (Rory A J Curtis).

ABCG4 gene expression was found to be regulated by oxysterols and retinoids in a similar manner to ABCG1 (Engel T et al., 2001). Both mouse ABCG1 and ABCG4 mediated cholesterol efflux to high density lipoproteins (Wang N et al., 2004). The human ABCG4 mRNA was found to be expressed primarily in brain and eye (Olsfield S et al., 2002; Annilo T et al., 2001; US2003/0166885). It was also suggested that ABCG4 is expressed at a high level in liver (Dean M et al., 2001).

It was observed that both transporter mRNAs are upregulated upon stimulation of sterol pathways. ABCG1 and ABCG4 were also associated with brain related disorders. In US200210169137 and in WO02/064781 (both by Reiner P B et al) it is experimentally supported that increased expression of functional ABCG1 and ABCG4 (and other ABC transporters) increases expression of Amyloid Precursor Protein (the precursor of β-amyloid, the component of amyloid plaques in Alzheimer's disease) in brain cells. In US2002/0192821 and in WO021094378 (Reiner, P B et al.,) is suggested that increased activity or expression of ABCG4 and other ABC transporter reduce catecholaminergic cell toxicity mediating e.g. Parkinson's disease.

Despite an extensive effort in the art to characterize the biological function of ABCG1 and ABCG4, no selective substrate or inhibitor of these proteins have been found. In fact, screening assays for identifying modulators of these transporter proteins are suggested in e.g. US200210169137, US2003/0027259 and in US2003/0166885 and described in generalized terms. However, the art is silent about a method for identifying modulators selective either for ABCG1 or ABCG4.

It is an object of the invention to provide methods for identifying selective modulators and methods for selective detection of ABCG1 and ABCG4.

To the best of our knowledge, we were the first to provide evidence that ABCG1 and ABCG4 can act both as homodimers and as heterodimers. Moreover, no isolated ABCG1/ABCG4 heterodimers have been provided before the advent of the present invention. Furthermore, no antibodies selective either to ABCG1 or ABCG4 have been provided in the art and no selective detection methods have been known to detect or quantify the present proteins or their activities.

In the present invention we expressed ABCG1, ABCG4 and their catalytic site mutants alone, and in various combinations, utilizing the Sf9 insect cells expression system. We prepared selective polyclonal and monoclonal antibodies that distinguish ABCG1 and ABCG4 e.g. in Western analysis; these antibodies allowed us to monitor the expression levels of both transporters in Sf9 membranes. In isolated membrane preparations we studied the vanadate-sensitive ATPase activity and screened over 100 compounds to stimulate or inhibit activity, in particular ATPase activity of the expressed proteins. By using the catalytic site mutants we could analyze the specificity? of the observed ATPase activities and their stimulation by putative transported substrate compounds. Moreover, by combined expression of the ABCG1, ABCG4 and their mutant variants we analyzed the possible dominant negative effects of the co-expressed proteins. Our data surprisingly show that both ABCG1 and ABCG4 are active and they function both as homo- and heterodimers in membranes. This first functional expression and characterization of ABCG1 and ABCG4 as interacting proteins may “fuel the fire” in the hunt for the physiological function of these proteins.

### BRIEF DESCRIPTION OF THE INVENTION

Screening Methods

The invention relates to a method for determining whether a substance is an activator, an inhibitor or a substrate of an ABCG1/ABCG4 heterodimer protein, comprising the steps of

providing the ABCG1/ABCG4 heterodimer protein in an active form,

contacting the heterodimer protein with the substance under conditions ensuring that the heterodimer protein exhibits detectable activity,

assessing activity of the heterodimer protein in the presence and in the absence of the substance, wherein

if, in the presence of the substance, the activity of the heterodimer protein is increased, the substance is considered as an activator of the ABCG1/ABCG4 heterodimer protein,

if, in the presence of the substance, the activity of the heterodimer protein is decreased, the substance is considered as an inhibitor of the ABCG1/ABCG4 heterodimer protein,

if the activity assessed is transport activity and the substance is transported by said heterodimer protein, the substance is considered as a substrate of the ABCG1/ABCG4 heterodimer protein.

The invention further relates to a method for determining whether a substance is a selective activator, a selective inhibitor or a selective substrate of an ABCG1/ABCG4 heterodimer protein, comprising the steps of

providing an ABCG1 homodimer protein, an ABCG4 homodimer protein and the ABCG1/ABCG4 heterodimer protein in active form,

the homodimer proteins and the heterodimer protein are separately contacted with the substance under conditions appropriate for detecting activity of the proteins,

assessing activity of the homodimer proteins and of the heterodimer protein in the presence and in the absence of the substance, wherein

if, in the presence of the substance, the activity of the heterodimer protein is increased whereas the activity of the homodimer proteins is not increased or increased only to a significantly lesser extent, the substance is considered as a selective activator of the ABCG1/ABCG4 heterodimer protein,

if, in the presence of the substance, the activity of the heterodimer protein is decreased whereas the activity of the homodimer proteins is not decreased or decreased only to a significantly lesser extent, the substance is considered as a selective inhibitor of the ABCG1/ABCG4 heterodimer protein,

if the activity assessed is transport activity and the substance is transported by said heterodimer protein whereas it is not transported by the homodimer proteins or transported only to a significantly lesser extent, the substance is considered as a selective substrate of the ABCG1/ABCG4 heterodimer protein.

In an embodiment, the invention relates to a method for determining whether a substance is a selective activator of an ABCG1 homodimer protein, an ABCG4 homodimer protein or an ABCG1/ABCG4 heterodimer protein, comprising the steps of

providing, in active form, at least two, preferably all the three dimer proteins of the following group: an ABCG1 homodimer protein, an ABCG4 homodimer protein and an ABCG1/ABCG4 heterodimer protein,

the proteins are separately contacted with the substance under conditions appropriate for detecting activity of the proteins,

assessing activity of the proteins in the presence and in the absence of the substance, wherein

if, in the presence of the substance, the activity of any one of the ABCG1 homodimer protein, the ABCG4 homodimer protein or the ABCG1/ABCG4 heterodimer is increased whereas the activity of the other two proteins is not increased or increased only to a significantly lesser extent, the substance is considered as a selective activator of the protein the activity of which is increased to the largest extent.

In an embodiment, the invention relates to a method for determining whether a substance is a selective inhibitor of an ABCG1 homodimer protein, an ABCG4 homodimer protein or an ABCG1/ABCG4 heterodimer protein comprising the steps of

providing, in active form, at least two, preferably all the three dimer proteins of the following group: an ABCG1 homodimer protein, an ABCG4 homodimer protein and an ABCG1/ABCG4 heterodimer protein,

the proteins are separately contacted with the substance under conditions appropriate for detecting activity of the proteins,

assessing activity of the proteins in the presence and in the absence of the substance, wherein

if, in the presence of the substance, the activity of any one of the ABCG1 homodimer protein, the ABCG4 homodimer protein or the ABCG1/ABCG4 heterodimer is decreased whereas the activity of the other protein(s) is not decreased or decreased only to a significantly lesser extent, the substance is considered as a selective activator of the protein the activity of which is decreased to the largest extent.

In an embodiment, the invention relates to a method for determining whether a substance is a selective substrate of an ABCG1 homodimer protein, an ABCG4 homodimer protein or an ABCG1/ABCG4 heterodimer protein comprising the steps of

providing, in active form, at least two, preferably all the three dimer proteins of the following group: an ABCG1 homodimer protein, an ABCG4 homodimer protein and an ABCG1/ABCG4 heterodimer protein,

the proteins are separately contacted with the substance under conditions appropriate for detecting transport activity of the proteins,

assessing activity of the proteins in the presence and in the absence of the substance, wherein

if the activity assessed is transport activity and the substance is transported by any one of the ABCG1 homodimer protein, the ABCG4 homodimer protein or the ABCG1/ABCG4 heterodimer whereas it is neither transported by the other protein(s) or transported only to a significantly lesser extent, the substance is considered as a selective substrate of the protein having the highest transport activity.

In a further preferred embodiment of this method

if, in the presence of the substance, both the activity of the ABCG4 homodimer protein and of the ABCG1/ABCG4 heterodimer protein is increased whereas the activity of the ABCG1 homodimer protein is not increased or increased only to a significantly lesser extent, the substance is considered as a selective activator of the ABCG4 protein,

if, in the presence of the substance, both the activity of the ABCG4 homodimer protein and of the ABCG1/ABCG4 heterodimer protein is decreased whereas the activity of the ABCG1 homodimer protein is not decreased or decreased only to a significantly lesser extent, the substance is considered as a selective inhibitor of the ABCG4 protein,

if the activity assessed is transport activity and the substance is transported by said ABCG4 homodimer protein and by the ABCG1/ABCG4 heterodimer protein, is whereas it is not transported by the ABCG1 homodimer protein or transported only to a significantly lesser extent, the substance is considered as a selective substrate of the ABCG4 protein.

In a further embodiment, analogously, i.e. by the very same method, the substances are tested to determine whether they are selective activator, a selective inhibitor or a selective substrate of an ABCG1 protein, preferably of an ABCG1 homodimer protein.

Preferably, in the method of the invention the assessed activity is ATPase activity. In the case of transported substrate, however, at least transport activity should advisably be assessed.

Preferably, the proteins are provided in cells or cell membrane preparations wherein it is ensured that no interfering ABC transporter activities are present, or at least it is ensured that the results are corrected for any interfering ABC transporter activities. Preferably it is ensured that no further, even potential dimerization partner is present.

With this proviso, the proteins can be provided in mammalian cells, preferably nerve cells (e.g. brain cells), immune cells, e.g. blood cells, e.g. macrophages, hepatocytes, kidney cells or epithel cells, or any other cells suitable to express said proteins or cell lines derived therefrom, or in mammalian cell membrane preparations. Preferably, the proteins are produced in said cells by recombinant expression.

In a preferred embodiment, the proteins may be expressed in yeast cells.

Highly preferably, the proteins are provided in insect cells.

A preferred expression system is the well-established Sf9-baculovirus expression system.

In preferred embodiments, the proteins are provided in membrane preparations, in particular membrane vesicles, preferably insect cell membrane preparations.

In a further preferred embodiment, any of the proteins is an active mutant of the corresponding wild type counterpart. As a control or in a dimer used as a control inactive mutants can be used. The mutant can be e.g. an appropriate active site mutant or a mutant having mutation in any of the Walker motifs.

Preferably, as an activity, at least ATPase activity, e.g. vanadate sensitive ATPase activity of the proteins is assessed; and/or membrane transport activity, e.g. direct transport of fluorescent compounds, e.g. Rhodamine derivatives, or labeled compounds is assessed.

Preferably, the substance is an anticancer agent, a receptor or channel modifier, a hormone, a neurotransmitter, a conjugate, e.g. glutathione conjugate or a conjugated bile acid, an ionophore, a peptide, a sterol, a dye, an amino acid, a peptide, a lipid, etc. or a derivative thereof. In an embodiment, the substance is a dye, preferably a rhodamine dye, a hormone, e.g. a thyroid hormone, a neurotransmitter, a neuropeptide, or a derivative thereof. In a further embodiment, the substance is a lipid, a sterol, e.g. a cholesterol or a molecule of the lipid or sterol metabolism or a derivative thereof, e.g. a labeled derivative.

The invention relates to the use of selective activators identified in the method of the invention as an activator of ABCG1 or ABCG4. Preferably the invention relates to the use of e.g. a rhodamine dye, preferably rhodamine 123 and rhodamine6G as a selective activator of an ABCG1 protein.

The invention also relates to the use of selective inhibitors, identified in the method of the invention as an Inhibitor of ABCG1 or ABCG4. Preferably the invention relates to the use of e.g. a benzamil or a benzamil derivative, a cyclosporin, preferably cyclosporin A or a thyroid hormone, preferably L-thyroxine as an inhibitor of ABCG1 protein in the method of the invention.

Heterodimer

In a further aspect, the invention relates to an isolated ABCG1/ABCG4 heterodimer protein.

Preferably, said heterodimer protein is present in a membrane of a cell, e.g. is a recombinantly expressed protein. The isolated ABCG1/ABCG4 heterodimer proteins can be present in a membrane preparation.

Membrane Preparations

The invention further relates to cell membrane preparations comprising at least one of the following isolated proteins: ABCG1 homodimer, ABCG4 homodimer, ABCG1/ABCG4 heterodimer. The membrane preparation of the invention is preferably a mammalian cell membrane preparation, an insect cell membrane preparation or a yeast cell membrane preparation, preferably a membrane vesicle preparation. In a highly preferred embodiment Sf9 membrane preparations or vesicles are applied.

Methods for the Preparation of Antibodies

The invention relates to a method for the preparation of an antibody selective for ABCG1 or ABCG4, wherein

N-terminal soluble domain of either ABCG1 or ABCG4 is expressed,

the protein is purified, and optionally pulverized and dried

the purified protein is mixed with adjuvant and injected into animals,

if desired the animals are boosted

sera are recovered,

the polyclonal antibodies obtained are checked for selectivity for ABCG1 or ABCG4, respectively,

if desired, monoclonal antibodies are prepared by usual means.

Preferably, the N-terminal soluble domain expressed contain at least the ATP-binding domain of either ABCG1 or ABCG4, preferably comprises amino acids 1-418 for ABCG1 and amino acids 1-386 for ABCG4, or an at least 100, preferably at least 200 amino acid fragment thereof.

The proteins are preferably expressed in bacteria and may form inclusion bodies, and

preferably expressed as a part of a function protein wherein the transporter sequence is fused to the C-terminus of an appropriate tag sequence, e.g. GST tag.

Antibodies

The invention further relates to an antibody selective for ABCG1, or an antibody selective for ABCG4.

The antibodies of the invention are directed to the ATP-binding domains of the proteins.

Said antibodies can be either polyclonal or monoclonal. Preferably, the antibodies are monoclonal.

Preferably, the antibodies of the invention are obtainable by the method of the invention for the preparation of antibodies.

Methods for Detection

The invention further relates to a method for detection of ABCG1 or ABCG4 protein in a biological sample, comprising the steps of

contacting the biological sample with an antibody selective for ABCG1 or ABCG4,

detecting binding of said antibody to the ABCG1 or ABCG4 proteins.

The invention further relates to a method for detection of ABCG1/ABCG4 heterodimers in a biological sample, comprising the steps of

contacting the biological sample with a reagent, preferably an antibody selective for ABCG1/ABCG4 heterodimer,

detecting binding of said reagent, preferably antibody to the heterodimer.

In a preferred embodiment, the invention relates to a method for detection of ABCG1/ABCG4 heterodimer proteins in a biological sample, comprising the steps of

contacting the biological sample both with an antibody selective for ABCG1 and with an antibody selective for ABCG4,

detecting binding events when both antibodies bind to the same heterodimer protein molecule.

Preferably, the antibody selective for ABCG1 and the antibody selective for ABCG4 comprise means for detection of proximity.

In a preferred embodiment, before contacting the sample with the antibody, at least a separation step is carried out so that the proteins of the sample are separated. If desired, the separated proteins are blotted to an appropriate membrane, and the antibody (antibodies) are added to this membrane as a contacting step and detection is carried out thereafter.

Methods for Modulating Function, Mutants

In a further aspect the invention relates to a method for modulating the function or activity of an ABCG1 and/or an ABCG4 homodimer protein and/or an ABCG1/ABCG4 heterodimer protein. This method comprises the step of substituting at least one of the subunits of said protein with e.g.

a mutant subunit of either ABCG1 or ABCG4, or

an ABCG4 subunit, or a mutant thereof, if the protein is ABCG1, or

an ABCG1 subunit, or a mutant thereof, if the protein is ABCG4,

wherein said mutant may be an inactive or an active mutant, e.g. a mutant of decreased or increased activity.

and optionally detecting an alteration in the function or activity caused by the said substitution.

The invention also relates to a method for preparing a mutant ABCG1/ABCG4 heterodimer protein, wherein

a mutant ABCG4 subunit and a wild type ABCG1 subunit is co-expressed, or

a mutant ABCG1 subunit and a wild type ABCG4 subunit is co-expressed,

a mutant ABCG4 subunit and a wild type ABCG1 subunit is co-expressed

in an appropriate host.

In a further preferred embodiment the mutant subunit, upon dimerization, results in an inactive protein useful e.g. as a control protein in the methods of the invention. The mutant can be e.g. an appropriate active site mutant or a mutant having mutation in any of the Walker motifs.

In a further preferred embodiment the mutant subunit is a subunit wherein activity of the protein, upon dimerization, is maintained.

The invention further relates to a method for detecting the presence of ABCG1 or ABCG4 homodimer or ABCG1/ABCG4 heterodimer activity in a biological sample comprising

obtaining a biological sample from a subject,

contacting the biological sample with a substance detectable as a selective activator or inhibitor of the ABCG1 or ABCG4 homodimer or ABCG1/ABCG4 heterodimer in a method of the present invention,

detecting an alteration in any activity attributable to the ABCG1 or ABCG4 homodimer or ABCG1/ABCG4 heterodimer specifically caused by the said activator or inhibitor, wherein said alteration is indicative of the presence of said ABCG1 or ABCG4 homodimer or ABCG1/ABCG4 heterodimer activity in said biological sample.

The invention further relates to a method for detecting the presence of ABCG1 or ABCG4 homodimer or ABCG1/ABCG4 heterodimer activity in a biological sample comprising

obtaining a biological sample from a subject,

contacting the biological sample with a substance detectable as a selective substrate of the ABCG1 or ABCG4 homodimer or ABCG1/ABCG4 heterodimer in a method of the present invention,

detecting transport activity attributable to the ABCG1 or ABCG4 homodimer or ABCG1/ABCG4 heterodimer specifically caused by the said substrate, wherein said activity is indicative of the presence of said ABCG1 or ABCG4 homodimer or ABCG1/ABCG4 heterodimer activity in said biological sample.

The invention further relates to a method for quantitating ABCG1 or ABCG4 homodimer or ABCG1/ABCG4 heterodimer activity in a biological sample comprising

obtaining a biological sample from a subject,

contacting the biological sample with a substance detectable as a selective activator or inhibitor of the ABCG1 or ABCG4 homodimer or ABCG1/ABCG4 heterodimer in a method of the present invention,

measuring an alteration in any activity attributable to the ABCG1 or ABCG4 homodimer or ABCG1/ABCG4 heterodimer specifically caused by the said activator or inhibitor, if desired by comparison with an appropriate control sample,

wherein the measure of said alteration is indicative of the level of the ABCG1 or ABCG4 homodimer or ABCG1/ABCG4 heterodimer activity in said biological sample.

The invention further relates to a method for quantitating ABCG1 or ABCG4 homodimer or ABCG1/ABCG4 heterodimer activity in a biological sample comprising

obtaining a biological sample from a subject,

contacting the biological sample with a substance detectable as a selective substrate of the ABCG1 or ABCG4 homodimer or ABCG1/ABCG4 heterodimer in a method of the present invention,

measuring transport activity attributable to the ABCG1 or ABCG4 homodimer or ABCG1/ABCG4 heterodimer specifically caused by the said activator, inhibitor or substrate, if desired by comparison with an appropriate control sample,

wherein the measure of said alteration is indicative of the level of the ABCG1 or ABCG4 homodimer or ABCG1/ABCG4 heterodimer activity in said biological sample.

In the present invention, the ABCG1 and/or the ABCG4 proteins herein are of vertebrata origin, preferably are of mammalian origin. The said transporter proteins of the invention are preferably rabbit, goat, sheep, pig or bovine, more preferably murine (rat or mouse) proteins. Highly preferably, the transporter proteins are human proteins.

### BRIEF DESCRIPTION OF THE FIGURES

FIG. 1.

A. Membrane topology and phylogenetic free model for the half-transporter ABCG family The NBD is located N-terminally (H₂N) to the TMD (proximal to the COOH end). The six membrane-spanning helicies (grey gradient) of the TMD are shown as cylinders passing through the lipid bilayer. A, B and C, mark the ATPase catalytic Walker A, Walker B and the Signature motifs, respectively. The KM arrow marks the catalytic site mutation (KM) engineered into the Walker A motif. The phylogenetic tree (bottom), comparing human ABCG family members, show that ABCG1 and ABCG4 are more closely related to each other than to ABCG2, the next most related member.

B. Western blot analysis of Sf9 expressed ABCG family members used in this study Membrane fractions (20 μg membrane protein), dissolved in disaggregation buffer, were separated on a 7.5% Laemmli-type gel and blotted onto PVDF. Filters were probed with anti-G1, anti-G4 or anti-G2 polyclonal antisera. ABCG1 (G1), ABCG1_{K124M} (G1_{KM}), ABCG4 (G4), ABCG4_{K108M} (G4_{KM}), co-expressed ABCG1 and ABCG4 (G1/G4), ABCG2 (G2) and control membranes from β-Gal virus infected cells.

FIG. 2. ATPase Activities Measured for ABCG1 and ABCG4 in Sf9 Membranes

ATPase activity of isolated Sf9 membranes was determined by measuring vanadate-sensitive inorganic phosphate liberation, using 3.3 mM MgATP. All measurements represent mean±SEM of the vanadate-sensitive ATPase activity in nmol Pi/min/mg membrane protein and are referred to as units (A). ATPase activities of membranes containing ABCG1 (G1), ABCG4 (G4), and ABCG2_{R482G} (G2_{G}), are shown as black bars, the corresponding KM mutants, ABCG1_{K124M} (G1_{KM}), ABCG4_{K108M} (G4_{KM}), and ABCG2_{R482G, K86M} (G2_{GKM}) are represented by white bars, whereas the background ATPase activity of β-Gal is shown as hatched bar and corresponding horizontal line. The asterisks denote ATPase activities statistically different from β-Gal activity (p<0.001). (B-F). Rhodamine123 stimulation Sf9 membranes containing ABCG1 and ABCG4 (B) ATP-dependence (C) and inhibition of ABCG1 activity by Benzamil (D), Cyclosporin A (E.), and L-thyroxin (F.). The ATPase activity of ABCG1 in the presence and absence of rhodamine123 is plotted as black up-triangles/solid lines and open circles/dashed lines. Open squares and solid line is ABCG4, solid squares and dotted line represent ABCG1_{K124M} whereas straight, dotted, line is β-Gal.

FIG. 3. ABCG1/ABCG4 Co-Expression ATPase Activity in SF9 Membranes

Sf9 cells were co-infected with ABCG1 plus β-Gal (G1+β-Gal), ABCG1 plus ABCG4_{K108M} (G1+G4_{KM}) and ABCG1 plus ABCG2_{R482G, K86M} (G1+G2_{KM}) (A); ABCG1 alone, ABCG4 alone and ABCG1 plus ABCG4 viruses. Membranes were isolated and ATPase assays were performed (A and C). Expression levels of ABCG1, ABCG4 and ABCG2, as well as of the inactive G4_{KM} mutant were determined with selective antibodies (B and D).

A. Membranes for each group, expressing similar levels of ABCG1, were used in ATPase assays. Black bars show basal activity and white bars show the rhodamine 123 stimulated (100 μM) activities. The solid, horizontal, line represents the β-Gal basal activity. Units are defined in FIG. 2A legend. Values are the mean±SEM for one experiment done in triplicate.

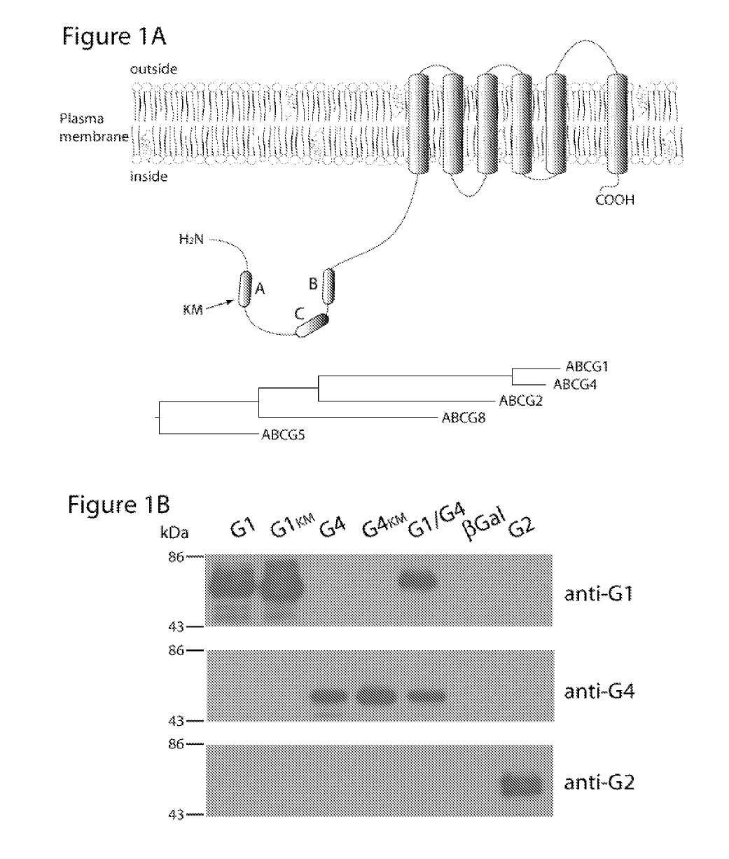

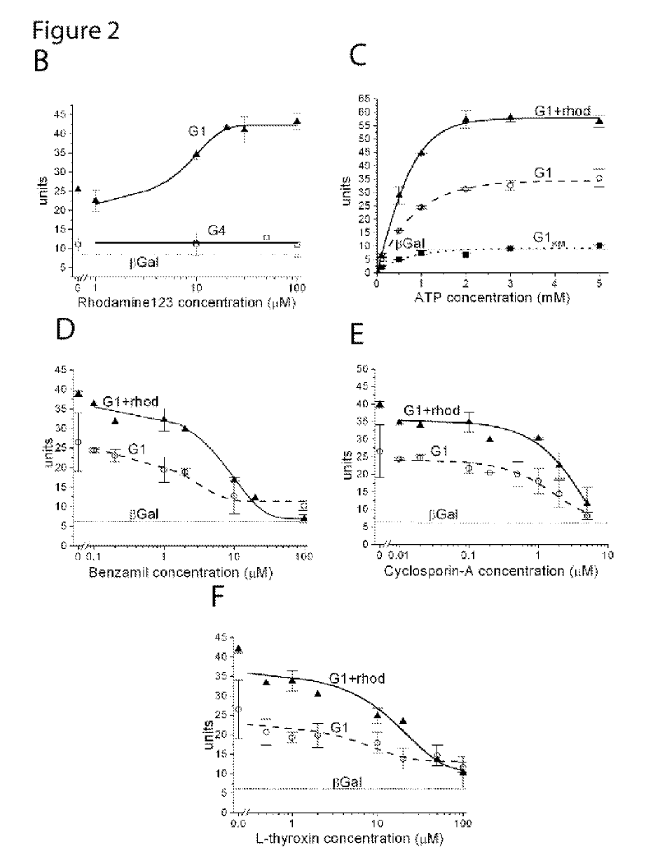

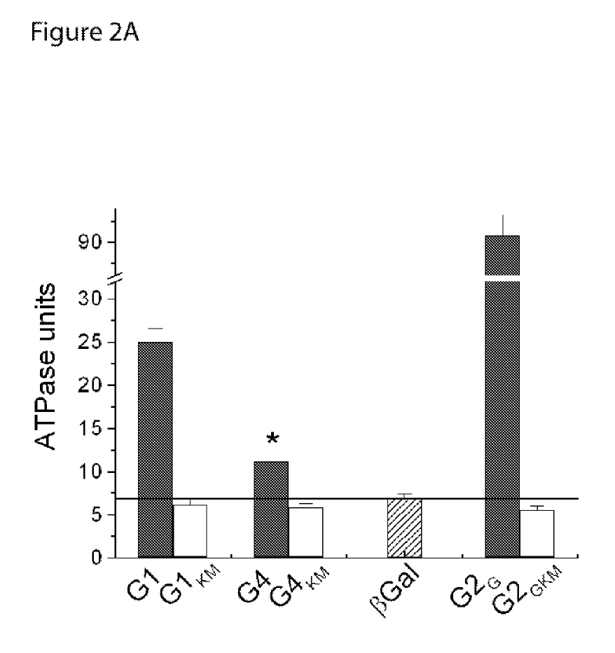

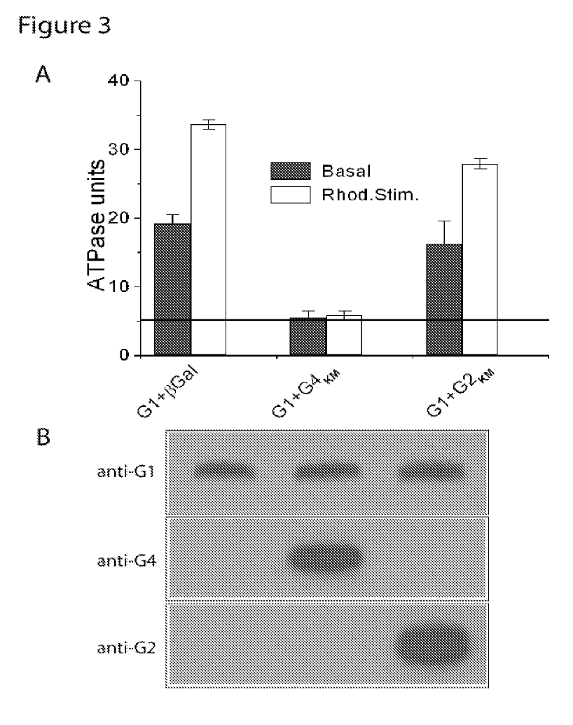

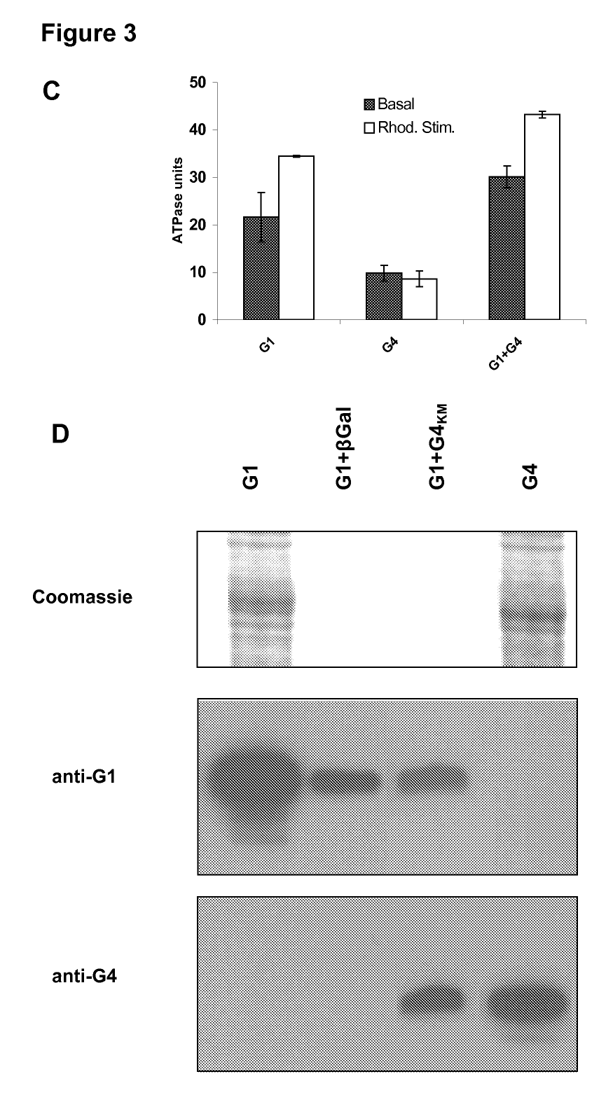

B. To ensure that a similar level of ABCG1 was expressed in each co-expression experiment, equal amounts of membranes (5 μg) were loaded onto 10% SDS-PA gels, electro-blotted and analyzed as described for FIG. 1B, using the same antibodies.

C. See A.

D. In order to demonstrate the similar expression level of G1 and G4_{KM} in the G1+G4_{KM} co-expressing membranes, the protein levels were compared with membranes expressing G1 or G4 alone. The Coomassie stained gels showed the same expression levels in case of G1 or G4 containing membranes. The immunoblot demonstrates the G1+G4_{KM} co-expressing membranes containing lower but similar amount of proteins.

### DEFINITIONS

The denomination “ABCG1” or “ABCG4” relates to human ABCG1 or ABCG4 proteins as well as their any mammalian counterparts or homologues or variants (Oldfield S et al., 2002, Annilo T et al., 2001), e.g. allelic variants (e.g. as disclosed in US2003/0027259), sequence variants (e.g. as disclosed in US2003/0166885) or splice variants occurring in nature. The denominations cover any functional mutants of the ABCG1 or ABCG4 proteins, preferably having at least 70, 73, 75, 78, 80, 83, 85, 88, 90, 92, 94, 95, 96, 98% sequence identity to any of the respective wild type counterparts. Sequence identity and percentage of sequence identity are well-known terms of the art and can be defined e.g. as in US2002/0169137, page 9 and 10. The denominations ABCG1 or ABCG4 can be used for monomeric or dimeric forms of said proteins as well as a subunit thereof, as specified by the context. Exemplary ABCG4 sequences are given e.g. in SwissProt at entry No Q9H172 (Homo Sapiens) and ABCG1 sequences are given e.g. in P45844 (Homo Sapiens) and Q64343 (Mus musculus).

A “homodimer” protein consists of two identical subunits whereas a “heterodimer” protein consists of two different subunits. It is to be understood that both homodimers and heterodimers may form larger oligomer complexes comprising multiple dimers (homo- or heterooligomers or -multimers). Thus, an oligomer consisting of only homodimers is comprised of an even number of the same monomers. Analogously, an oligomer consisting of only heterodimers is comprised of two type of monomer subunits forming heterodimers with each other. Mixed oligomers, comprising homodimer(s) and heterodimer(s) at the same time may also exist.

An “ABCG1/ABCG4 heterodimer” is a dimeric protein on of the subunit of which a an ABCG1 subunit and the other is an ABCG4 subunit, either a wild type or a mutant thereof, as explained above.

The term “isolated” is meant herein as “its natural environment has been changed by Man”. Thus, the environment of an “isolated protein” must be different from its natural environment. In particular, an isolated protein may be expressed in a host, e.g. a host cell transformed by the gene encoding said protein, said cell being incapable of expressing the protein originally; or an isolated protein may be removed from its original environment; or both. The isolated protein may then be processed further. A protein overexpressed in a cell in which said protein is expressed otherwise, i.e. of itself, is not considered herein as an isolated protein

The term “specific” is meant herein as “having distinctive property or character” or “having properties that allow distinguishing” or “capable of exerting a distinctive effect or influence”. A particular meaning of “specific” is “selective” and the latter term is used in the context of making a distinction between similar proteins. Thus, a selective activator or a selective inhibitor of a transporter protein refers to a substance having a significantly, e.g. detectably higher activating or inhibiting affect on the said transporter protein than on a transporter similar thereto. Analogously, a selective substrate of a protein is a substance which is a “better” substrate (i.e. is transported with higher activity or increases ATP-ase activity to a higher level) by the said transporter than by a transporter similar thereto. Similarly, an antibody selective for a given transporter protein is capable of binding to said transporter with an affinity higher than the binding affinity of the same antibody to an other transporter, thereby enabling selective detection of the said transporter protein. The transporter protein mentioned herein, depending on the context is preferably an ABCG1 protein or an ABCG4 protein, in particular an ABCG1 homodimer protein or an ABCG4 homodimer protein or an ABCG1/ABCG4 heterodimer protein.

The term “activity” of an ABC transporter protein refers to any activity exerted by the said transporter protein including e.g. its biological function, transport activity, i.e. transport of a drug through the membrane carrying the said protein, or ATP-ase activity etc. In a broad sense, “activity” also covers herein any partial reaction (e.g. substrate binding) of the whole reaction cycle of the enzyme as well as a partially damaged activity, e.g. ATP-binding, nucleotide occlusion (trapping), enabling detection of the function, e.g. a cell biological effect, of the enzyme. An “activator” substance increases activity, whereas an “inhibitor” substance decreases activity of the said ABC transporter.

“Functional fragments” of ABCG1 and ABCG4 half transporter proteins are fragments of the proteins maintaining at least their dimerization property and, preferably, at least partial activity of the said protein.

The abbreviations used: ABC, ATP binding cassette; wt, wild-type; Sf9, Spodoptera frugiperda

Further terms and abbreviations are used as accepted in the art.

### DETAILED DESCRIPTION OF THE INVENTION

The invention is disclosed by way of illustrative examples in more detail below. It is to be understood that the examples are not intended to limit the claimed scope of the invention. It should also be kept in mind that the skilled person is able to modify the solution disclosed herein or create variants thereof without departing from the spirit and scope of the invention.

In this study we utilized a baculovirus Sf9 insect cells system to express and biochemically characterize the human ABCG1 and ABCG4 transporters. Heterologous baculovirus expression allows high level and “fine-tuning” of the transporter protein(s) expressed, without the possible interference of endogenous mammalian type ABC transporters. This methodology has been used earlier in our laboratory to characterize several transporters of the MDR, MRP, and ABCG family.

For this work we also developed for the first time selective polyclonal antisera that recognize and distinguish the highly similar (72% identity at the amino acid level) ABCG1 and ABCG4 proteins, allowing detection of their expression in Sf9 membranes. From the polyclonal antisera we have developed monoclonal antibodies selective for ABCG1 or ABCG4 transporters.

ABCG half-transporters function either as homodimers (ABCG2, Mimoto et al., 2003, Özvegy C et al., 2002) or heterodimers (ABCG5 and ABCG8, Graf G A et al., 2003). The question whether ABCG1 and ABCG4, in particular human ABCG1 and ABCG4 act as homodimers or heterodimers and, if the latter is the case, they form heterodimers with each other or with potential unknown partners, has been highly debated but not decided in the art. Since Drosophila homologues (White, Scarlet and Brown), which are rather distant relatives of the human ABCG1 and ABCG4, as well as human ABCG5 and ABCG8 appear to function as a heterodimer (Haimeur A, 2004, Graf G A et al., 2003) it has been proposed that ABCG1 and ABCG4 may do the same (Annilo T et al., 2001, Graf G A et al., 2003). This idea seemed to be supported by the results that both transporters is up-regulated in response to LXR and RXR agonists (Laffitte B A et al., 2001, Engel T et al., 2001). However, their dissimilar tissue distribution (see e.g. Klucken J 2000, Oldfield S 2002, Annilo T 2001), as well as transient overexpression of murine ABCG1 and ABCG4 in mammalian cells by Wang N et al., (2004) seemed to contradict to the heterodimer theory.

In the present invention we used insect cells to avoid any disturbing affect of a potential unknown dimerization partner and succeeded in detecting functional ABCG1 and ABCG4 homodimers by measuring ATPase activity of the proteins. It appeared that the long-existing uncertainty in the art bad been removed. Any half transporters known so far were found to be acting either as homodimers or as heterodimers.

However, when co-expressing the catalytic site mutant ABCG4_{K108M} with functional ABCG1, we observed a dominant-negative effect of the mutant ABCG4 on ABCG1 activity (FIG. 3). In contrast, the ABCG2 catalytic site mutant, when co-expressed with ABCG1, had no effect. We conclude that this is due to a specific interaction of ABCG4 with ABCG1 in a heterodimeric complex. Catalytic site mutations (KM) in the Walker A motif of ABCG1, ABCG4 or ABCG2 rendered the homodimeric transporters inactive. We previously reported that mutating just one Walker A motif in a full transporter, like MDR1, is enough to inactivate the transporter (28). In line with this, we now show that one functional ABCG1 subunit interacting with a catalytically inactive ABCG4_{K108M} subunit is not active as a whole. In summary, our data indicate that ABCG1 and ABCG4 can form both homo- and heterodimers.

The above results enabled us to devise screening methods for identifying selective modulators, in particular activators, inhibitors or substrates of ABCG1 or ABCG4 homodimers or for ABCG1/ABCG4 heterodimers. Antibodies of the invention and selective modulators identified by the methods allow detection, identification and quantitation of these proteins in various tissues and biological samples.

In the method of the invention we also identified rhodamine123 as an ATPase activator and thus potential substrate for ABCG1. Moreover, by screening a large compound library, we found several agents which strongly inhibited ABCG1 ATPase activity at relatively low concentrations.

Though expression of the proteins of invention in insect cells was important to obtain unambiguous results to answer the question of homodimerization or heterodimerization, it is to be understood that in the screening methods of the invention the proteins can be expressed in other cell lines suitable for expressing the half transporters of the invention, provided that it is confirmed that homodimers and heterodimers of the invention are present. In the light of the present disclosure this task is within the skills of a person skilled in the art. For example, if the half transporters of invention are expressed at various levels (expression levels can be assessed by using the selective antibodies of the invention) and activities measured are directly proportional to the expression levels, it indicative of the fact that dimers are formed of the half transporters expressed.

Examples of mammalian cells appropriate for the present invention are nerve cell lines (e.g. Neuro2a), blood cell lines (e.g. HL60), hepatocyte cell lines (e.g. HepG2), kidney cell lines (e.g. COS-7), epithel cell lines (HeLa).

As explained below, selective polyclonal antibodies were prepared by fusing the N-terminal soluble domain of each transporter, which contain the ATP-binding domains (amino acids 1-418 for ABCG1 and 1-386 for ABCG4), to the C-terminus of GST. Proteins have been expressed, pulverized, dried, mixed with adjuvant and injected into mice. Mice were boosted and sera were recovered for use in this study. Monoclonal antibodies have been prepared by usual methods. Thereafter selectivity of antibodies have been tested. It is to be understood, however, that any antibodies, either polyclonal or monoclonal against the N-terminal soluble domain of any variant or mutant of ABCG1 or ABCG4 is within the scope of the present invention. In particular, antibodies selective for either ABCG1 or ABCG4 are contemplated in the present invention. The antibodies can be any type of antibodies, including any isotype thereof. The antibodies can be e.g. humanized antibodies, CDR grafted antibodies etc. Antibody fragments, having the same complementarity determining regions (CDRs) as those of the antibodies of the invention are also contemplated herein.

The invention also relates to reagents capable of specific or selective recognition of ABCG1 or ABCG4, comprising CDRs of the antibodies of the invention.

The invention is illustrated further by the specific, non-limiting examples below.

Experimental Procedures

Materials

Rhodamine123, Rhodamine6G, Na-orthovanadate, 3-OH-kynurenine, cyclosporin A, benzamil, L-thyroxin, and ATP were from Sigma. Ko143 was a generous gift from Drs. J. Allen and G. Koomen (University of Amsterdam, Amsterdam, The Netherlands).

Generation of Baculovirus Vectors Expressing the cDNAs of Human ABCG1 and ABCG4

To construct a human ABCG1 expression vector, a 2038 nucleotide cDNA fragment of the long isoform of ABCG1 was amplified with primers ABCG1F (5′-caccatggcctgtctgatggccgc-3′) and ABCG1R (5′-tcctctctgcccggattttgtac-3′) by RT-PCR from macrophage cDNA and inserted into the pcDNA3.1/CT-GFP-TOPO vector (Invitrogen) by TA-cloning. Subsequent PCR subcloning placed the cDNA in the baculovirus expression vector, pAcUW21-L, and added a stop coding. ABCG4 cDNA was purchased from the I.M.A.G.E. consortium (clone ID 1537140). It was PCR cloned to add an A to the Start ATG at the 5′-end of the gene and cloned into pAcUW21-L as described elsewhere (Özvegy C et al., 2001). ABCG1 and ABCG4 cDNAs were sequenced to confirm no errors existed. Catalytic site mutants were prepared using the following PCR mutagenic primers: ABCG1: 5′-gcgtggacatgccggccc-3′ and 5′-gggccggcatgtccacgct-5′, and ABCG4: 5′-cgggagctgattggcatcatgggccc ctcaggggctggcatgtctac-3′ and 5′-ggctcatcaaagaacatgacaggcg-3′. Subsequent subcloning replaced the corresponding regions of wild-type constructs with PCR products carrying the mutation.

Generation of Polyclonal Antibodies Against ABCG1 and ABCG4 and Western Analysis

Polyclonal antibodies were prepared by fusing the N-terminal soluble domain of each transporter, which contain the ATP-binding domains (amino acids 1-418 for ABCG1 and 1-386 for ABCG4), to the C-terminus of GST. In detail, two DNA fragments encoding intracellular regions were PCR amplified from ABCG1 and ABCG4 cDNAs (the primers used 5′-atgcggatccccatggcctgtctgatggc-3′ and 5′-atgcctcgagtcacctcatgatgctgagg-3′ for ABCG1 and 5′-atgcgaattcatggcggagaaggcg-3′ and 5′-atgcgcggccgctcagaggatggacaggaaggtc-3′ for ABCG4). The PCR products were digested by BamHI/XhoI and EcoRI/NotI, respectively, and cloned into the pGEX 5x-1 vector (Amersham Biosciences).

Strain BL21(Stratagene) was transformed with the plasmids and protein expression was induced using IPTG at a final concentration of 1 mM. Upon bacterial harvest the insoluble membrane-containing fractions (inclusion bodies) were separated by differential centrifugation, sonicated and resuspended in PBS. Protein suspensions were solubilized in 0.1 M urea and sample buffer and resolved on 7.5% polyacrylamide gel. Bands corresponding to the fusion proteins were determined by Coomassie staining of parallel controls, and were cut from the gel. The gel slices were lyophilized, pulverized with a mortar and pestle, and resuspended in phosphate buffered saline. These preparations were emulsified in Freund's adjuvant and injected into mice. Mice were boosted and small amounts of sera were recovered for use in this study and tested by Western analysis as previously described (Sarkadi B et al., 1992). Secondary antibody was anti-mouse, peroxidase-conjugated, goat IgG (Jackson Immunoresearch), used in 10,000× dilutions.

Generation of Monoclonal Antibodies

Monoclonal antibodies were generated as follows: Mice possessing reactive serum were boosted with the purified protein and 3 days later were sacrificed and their spleen was removed. The splenocytes were fused with a myeloma cell line and plated in 96 well plates. Clones were screened by ELISA and immunoblot analysis.

Generation of Recombinant Baculoviruses, Expression in Sf9 Cells and ATPase Activity Measurements

Recombinant baculoviruses carrying transporter cDNA were generated with BaculoGold Transfection Kit (Pharmingen), in accordance with the manufacturer's protocol. The titer of virus supernatants were determined in order to express the same amount of each proteins, Sf9 (Spodoptera frugiperda ovarian) cells (Invitrogen) were infected and cultured according to the procedures described previously (19). Briefly, about 3×10⁷ cells were infected with 3 ml of virus supernatant in case of homodimer expression and 1.5-1.5 ml of virus supernatants in case of heterodimer expression. The virus-infected Sf9 cells were cultured in T150 culture flasks with 30 ml of medium for the times indicated. The cells were harvested by scraping them into Tris-mannitol buffer (50 mM Tris, pH 7.0, with HCl, containing 300 mM mannitol and 0.5 mM phenylmethylsulfonyl fluoride).

For membrane preparation the cells were lysed and homogenized using a glass-Teflon tissue homogenizer in TMEP (50 mM Tris, pH 7.0, with HCl, containing 50 mM mannitol, 2 mM EGTA-Tris, 10 pg/ml leupeptin, 8 pg/ml aprotinin, 0.5 mM phenylmethylsulfonyl fluoride, and 2 mM dithiothreitol), and the undisrupted cells and nuclear debris were removed by centrifugation at 500×g for 10 min. The supernatant fluid was then centrifuged for 60 min at 100,000×g and the pellet containing the membranes resuspended in TMEP at a protein concentration of 2-3 mg/ml. All procedures were carried out at 4° C., and the membranes were stored at −70° C.

The ATPase activity of the isolated Sf9 cell membranes was estimated by measuring inorganic phosphate liberation. Membrane suspensions (about 20 μg of membrane protein, as determined by a modified Lowry method) were incubated at 37° C. for 20-min in 0.15 ml of a medium containing 40 mM MOPS-Tris (pH 7.0), 0.5 mM EGTA-Tris (pH 7.0), 2 mM dithiothreitol, 50 mM KCl, 1 mM ouabain and 5 mM sodium azide, and the ATPase reaction was started by the addition of 3.3 mM MgATP. The indicated drugs (obtained from Sigma) were added in dimethyl sulfoxide. The final concentration of dimethyl sulfoxide in the assay medium was 1%. Control experiments indicated that dimethyl sulfoxide at this concentration had no appreciable effect on the ATPase activity. The reactions were stopped by the addition of 0.1 ml of 5% SDS solution and the amount of inorganic phosphate determined immediately. Inorganic phosphate was measured by colorimetric reaction (19). The points plotted in the figures indicate the means of triplicate determinations.

Results and Discussion

Topology Model and Computer Predictions

Human ABCG1 and ABCG4 are ABC half-transporters with similar length (ABCG1 and ABCG4 contain 678 and 646 amino acids, respectively) and share 72% amino acid sequence identity. The membrane topology of ABCG family transporters is assumed to be similar (the phylogenetic tree and the ABCG family structure is shown schematically in FIG. 1A). We modelled this structure using the online software program HMMTOP (http://www.enzim.hu/hmmtop/; Tusnady G E et al., 1998). The NBD (or ABC) is N-terminal to the TMD (FIG. 1A). Based on the topology model and computer predictions, neither ABCG1 nor ABCG4 have N-glycosylation receptor sites.

Expression of ABCG1 and ABCG4 and Their Catalytic Site Mutants

In order to biochemically characterize ABCG1 and ABCG4, we utilized the baculovirus-infected Sf9 cell system which had been used to successfully express biologically active ABCG2 at high levels in Sf9 cell membranes (Özvegy C et al., 2001). We generated recombinant baculoviruses containing the cDNAs of the ABCG family members. Viruses were propagated in Sf9 cells and high-titer viruses were produced and used to infect new cultures of Sf9 cells. Two days after transfection, the cells were harvested and membranes containing the transporter of interest were prepared.

As a control of activity, we generated catalytic site mutants by replacing the conserved lysine residue in the Walker-A region of ABCG1 and ABCG4 with methionine in both transporters (ABCG1_{K124M} and ABCG4_{K108M}—see FIG. 1A, KM and arrow pointing to the Walker A motif mutation) which we expected would abrogate ATPase activity as we also observed for ABCG2 (Özvegy C et al., 2002). It will be understood that for the same purpose any inactive mutant folded correctly is appropriate. Further Walker A and/or Walker B motif mutants are described e.g. in US2002/0169137, page 23 and 24.

To determine the background ATPase activity in the Sf9 cell membranes we produced β-Galactosidase (β-Gal) virus infected Sf9 membranes. For comparison, we chose to express the glycine variant, ABCG2_{R482G}, (and its catalytic site mutant ABCG2_{R482G, K86M}), which is well characterized and transports rhodamine123 (Özvegy C et al., 2001) as we found for ABCG1 (see below, Özvegy C et al., 2002). All transporters were expressed at high level and their presence was observed by Coomassie staining (FIG. 3b) and Western analysis (see below).

Detection of ABCG1 and ABCG4, Preparation of Selective Antibodies

In order to follow the expression of ABCG1 and ABCG4 and to distinguish these two closely related transporters, we produced polyclonal antibodies against the N-terminal soluble domain of each transporter (see Experimental Procedures). Quite unexpectedly, these antibodies proved to be selective in distinguishing ABCG1 and ABCG4 in Western blots (FIG. 1B). It is to be noted that due to the high sequence similarity of the two proteins preparation of selective antibodies proved to be a difficult task. Usual strategies based on selection of potential epitopes having relatively large differences in sequence have failed to produce selective antibodies. For example, when short peptide sequences were used for inmunisation (e.g. RKKGYKTLLKGISGK or KGISGKFNSG for ABCG1, KCLSGKFCRR for ABCG4) no selective antibody were obtained, and the generated antibodies recognized both proteins. Additionally, according to informal communication from other groups investigating these proteins, efforts to produce selective antibodies did not meet with success.

Thus, we applied a less promising method to produce selective antisera against the N-terminal soluble domain of each transporter, which contain the ATP-binding domains, with a surprising success.

We used these antibodies to follow the expression of ABCG1, ABCG1_{K124M}, ABCG4, ABCG4_{K108M}, ABCG2, β-Gal and the co-expressed ABCG1 and ABCG4, in isolated Sf9 cell membranes, by Western analysis. As documented in FIG. 1B, the anti-G1 antibody selectively recognized ABCG1 and ABCG1_{K124M} but not ABCG4, ABCG4_{K108M}, ABCG2 or any other Sf9 protein bands (anti-G1 panel). ABCG1 and ABCG1_{K124M} were expressed at high levels in the membrane; when ABCG1 was co-expressed with ABCG4, the ABCG1 level was reduced but still observed as a single band migrating at approximately 60 kDa. ABCG4 and ABCG4_{K108M} were selectively recognized migrating slightly faster than ABCG1, also at approximately 60 kDa, by the anti-G4 antibody (FIG. 1B, anti-G4 panel). Neither antisera recognized ABCG2 or other nonspecific bands in the control β-Gal lane. The anti-G2 monoclonal antibody, BXP-21, was specific (selective) for ABCG2 (FIG. 1B, anti-G2 panel). Two bands for ABCG2 were observed; the higher one may be the core glycosylated form of ABCG2 (Özvegy C et al., 2001). These selective antisera allowed us to fine-tune the levels of transporter expression in Sf9 cells.

From the polyclonal sera monoclonal antibodies have been developed by the method described in the “Experimental procedures”. We also prepared monoclonal antibodies selectively recognizing ABCG1 and ABCG4.

When activities of the proteins are compared, e.g. in the screening methods of the invention, it is advisable to use about the same expression levels. Control of expression levels may be particularly important when ABCG1 and ABCG4 monomers are co-expressed and the same expression level is to be achieved.

ATPase Activity of ABCG1 and ABCG4 in Sf9 Cell Membranes

Most ABC transporters bind and hydrolyze ATP, which provides the energy for transport. When expressed in Sf9 membranes, the function of several ABC transporters has been successfully examined by investigating the sodium orthovanadate sensitive and substrate-modified phosphate liberation in isolated membranes (Özvegy C et al., 2002, Özvegy C et al., 2001, Sarkadi B et al., 1992).

In order to characterize the function of ABCG1 and ABCG4 we subjected isolated Sf9 cell membranes containing these transporters to ATPase activity measurements. FIG. 2A shows that ABCG1 has a relatively high vanadate-sensitive basal ATPase activity of 25±1.57 (SEM, n=15) nanomoles Pi/mg membrane protein/min (defined as units), compared to background activity. The background ATPase activity for Sf9 membranes not expressing a heterologous transporter is low, as found in membranes of Sf9 cells expressing the β-Gal protein (6.8±0.56, SEM, n=15, units). The ABCG1_{K124M} catalytic site mutant has an activity of 6.1±0.83 (SEM, n=8) units which is similar to the background and therefore is considered inactive.

We measured the basal ATPase activity for ABCG4 and observed 11.1±0.95 (SEM, n=15) units activity. This ABCG4 activity was shown to be statistically different from the background (p<0.001, FIG. 2A, labeled by an asterisk). Although this basal activity is relatively small, it is similar to that found for some functional, transport competent ABC transporters, e.g. MRP6 and MRP3 (Ilias A et al., 2002, Bodo A et al., 2003). As expected, the ABCG4_{K108M} catalytic site mutant is inactive (5.8±0.51, SEM, n=3, units).

For comparison, we plotted ABCG2_{R482G} (G2_{G}; 90.84±2.49, SD, units) which was determined in our lab (Özvegy-Laczka, C., personal communication). The activity of the active site mutant ABCG2_{R482G,K86M} was similar to the background ATPase activity. These results are consistent to those published for ABCG2 and small differences could result from varying amounts of protein expressed in the Sf9 cells (Özvegy C et al., 2002).

ABCG1 and ABCG4 Form Homodimers when Expressed Separately in Sf9 Membranes

We found that these closely related human ABC half-transporters functioned as vanadate-sensitive membrane ATPases. Since dimerization is a requirement for the function of the G type ABC half transporters, this fact indicates that both ABCG1 and ABCG4 can work as homodimers.

Since expression studies have been carried out in Sf9 cells, it can be excluded that a further dimerization partner is present.

ABCG1 and ABCG4 form Heterodimers when Co-Expressed in, Sf9 Membranes

Since it has been proven above that ABCG1 and ABCG4 act as homodimers, it was expected that their subunits can not form heterodimers. In order to confirm this hypothesis we co-expressed ABCG1 in various combinations and assayed the basal and rhodamine123 stimulated ATPase activity in Sf9 membranes. We reasoned that the co-expression of ABCG1 with the non-functional catalytic site mutant, ABCG4_{K108M}, would not interfere with ABCG1 ATPase activity, but could result in lowered ATPase activity if the two proteins interacted to any extent.

In order to study the interaction between ABCG1 and ABCG4, ABCG1 was co-expressed with different viral quantities of β-Gal baculovirus, which allowed the normalization of ABCG1 expression per mg membrane protein. ATPase activity was measured for membranes expressing certain levels of ABCG1, as assayed by using the anti-G1 selective antibody. In similar experiments, ABCG1 was also co-expressed with ABCG4 or the ABCG4_{K108M} mutant protein, and the same enzymatic assays were performed, in membranes containing the same levels of ABCG1, as detected by Western blotting and subsequent signal densitometry analysis (FIG. 3B).

As shown in FIG. 3A, when co-expressed with β-Gal, ABCG1 had a basal and rhodamine123 stimulated activity of 19.2±1.24 (SEM, n=3) and 33.6±0.65 (SEM, n=3) units (FIG. 3A, G1+β-Gal). Surprisingly, the non-functional ABCG4_{K108M} mutant, when co-expressed with ABCG1, severely abrogated the ABCG1 activity over background (FIG. 4A, G1+G4_{KM}). The horizontal line through the bar graph represents the background (β-Gal) ATPase activity level observed for the experiment. This experiment, performed with similar levels of ABCG1 expression (see Panel B), shows that ABCG4_{K108M} can interact with ABCG1 in membranes and the mutant ABCG4 induces a dominant negative effect on ABCG1 ATPase activity.

In order to examine the specificity of the ABCG1-ABCG4 interaction, the catalytic site mutant ABCG2_{R482G, K86M} was also co-expressed with ABCG1. As documented, the ABCG2_{R482G, K86M} mutant did not interfere with ABCG1 activity (FIG. 3A, G1+G2_{KM}). As a further control, the ABCG4_{K108M} mutant was co-expressed with the functional ABCG2_{R482G}; however, no dominant-negative effect of ABCG4_{K108M} on the ATPase activity was observed (data not shown). Western blot analysis carried out with the selective antibodies of the invention confirmed the presence of ABCG4_{K108M} and ABCG2_{R482G, K86M} in these membrane preparations (FIG. 3B, anti-G4 and anti-G2).

A protein will not form a dimer with another protein unless it is its natural dimerization partner. For example, ABCG2_{R482G, K86M} does not interact with ABCG1 and does not affect its function. Thus, abrogation of ABCG1 function by the mutant, inactive ABCG4 is a clear indication of dimerization. It is generally accepted that alteration of function is stronger evidence for dimerization than binding methods or fluorescent excitation or quenching methods.

In an experiment ABCG1 and ABCG4_{K108M} have been co-expressed at about the same level (FIG. 3D). Though it could be expected that a certain amount of homodimer should be present dependent on affinity coefficients of formation of the various complexes, ABCG1 activity could not be detected. Therefore it is thought that affinity coefficient of the heterodimer is higher than that of the homodimers and, when co-expressed at about the same level in Sf9 cells, only small or negligible amount of homodimers are present.

Providing Active Heterodimers

In a similar experiment ABCG1 and ABCG4 were also co-expressed but no appreciable difference in the ABCG1 ATPase activity was detected (FIG. 3.C). When expression ratios were varied, if any of the dimerization partners was expressed in a significantly higher ratio, the activity obtained was similar to that of the respective homodimer.

It will be important to test the effects of co-expression of ABCG1 and ABCG4 in other systems and using (still unknown) physiological substrates of these proteins.

Screening of Various Compounds

In the above experiments is has been shown that ABCG1 and ABCG4 can form both homo and heterodimers. This finding could be utilized in screening methods for identifying selective modulators of any of the dimer types.

The ATPase activity measurements for ABCG1 and ABCG4 in isolated Sf9 cell membranes allowed us to search for substrates and inhibitors which could stimulate or inhibit this ATPase activity. To this end, we screened about 100 compounds for their ability to alter the ATPase activity of these transporters. These compounds fell into several categories: anticancer agents (e.g. mitoxantrone, doxorubicin), receptor/channel modifiers (e.g. benzamil), prostaglandins, kynurinins (e.g. 3-OH-kynurenine), hormones and neurotransmitters (e.g. L-thyroxine), conjugated bile-acids, glutathione conjugates, ionophores, peptides, sterols, fluorescent (e.g. rhodamine123, calcein-AM) and other small molecules (e.g. cyclosporine A, Ko134, verapamil). Surprisingly, we found that rhodamine123 (and to a lesser extent rhodamine6G, data not shown) could substantially stimulate the ATPase activity of ABCG1 in a concentration dependent (FIG. 2). In the presence of 20 μM rhodamine123, the ATPase activity of ABCG1 was increased almost two fold and we calculated a K_{act} for rhodamine123 of about 10 μM. The compound had no effect on ABCG4 ATPase activity (FIG. 2B). The effect of rhodamine123 on ABCG1_{K124M} was negligible (data not shown). It should be mentioned that rhodamine123 also stimulates the ABCG2_{R482G} mutant ATPase activity about 1.4-fold (Özvegy C et al., 2001).

The vanadate-sensitive basal ATPase activity of ABCG1 was relatively high and, similar to ABCG2 (Özvegy C et al., 2001), it appears that a yet unidentified compound in Sf9 membranes can stimulate ABCG1 activity. In fact, ABCG2 has been found to transport sterols in bacteria (Janvilisri T et al., 2003) and overexpression leads to the extracellular exposure of phosphatidylserine in cancer cells (Woehlecke H et al., 2003). Also, ABCG1 has been implicated in sterol transport (see introduction).

Basal ATPase activity could reflect a partial uncoupling of the ATPase function in unfolded transporter. To explore this, and to determine the correct working concentration of ATP in experiments, we measured the MgATP dependence on ABCG1 in the presence and absence of substrate. FIG. 2C shows the ABCG1 ATPase dependence on ATP with or without 100 μM rhodamine123, over a range of ATP concentrations. The calculated Kₘ for ATP was found to be approximately 0.5 mM in both cases. The ATP dependence of the ABCG1_{K124M} ATPase activity is also shown (dotted line). These data are consistent with those published for ABCG2_{R482G}, showing a Kₘ for ATP of about 0.6 mM (Özvegy C et al., 2001). Since the Km ATP for ABCG1 in the presence or absence of the stimulating compound, rhodamine123, are similar, we conclude that the majority of ABCG1 in Sf9 membranes is intact and that the high vanadate-sensitive basal ATPase activity observed for ABCG1 is brought about by molecules, possibly lipid or lipid derivatives, present in the membrane as previously proposed for ABCG2 (Özvegy C et al., 2001). ABCG4 activity was maximal at the concentrations used in these experiments (data not shown).

The search for compounds that interact with ABCG1 led to the identification of several inhibitors of ABCG1 function (FIG. 2D-F). These drugs inhibited both the basal and the rhodamine123 activated ATPase activity of ABCG1 but not the KM mutant (data not shown). Benzamil, a substrate of ABCG2, decreased the ABCG1 ATPase activity to the control level with a Kᵢ about 0.5 μM. Cyclosporin A, an inhibitor of ABCG2, also inhibited ABCG1 ATPase activity at low concentrations. Additionally, the thyroid hormone L-thyroxine also inhibited ABCG1. It is worth mentioning that the specific ABCG2 inhibitor, Ko143, had no effect on the activity of ABCG1 (data not shown).

ABCG4 had a low, but statistically significant level of ATPase activity, which could not be stimulated or inhibited by more than 100 potential substrate compounds tested. Though theoretically it may occur that the protein is not entirely folded in Sf9 cells and therefore can not be stimulated by substrate, it is more probable that ABCG4 is fully functional but we did not find a substrate that can stimulate its ATPase activity. In fact, the relatively low ABCG4 basal activity observed here is reminiscent of the activity found for functional, transport competent, MRP6 and MRP3 (Ilias A et al., 2002, Bodo A et al., 2003).

Modulators of the ABCG1/ABCG4 heterodimer can be tested the same way as those of the homodimers described above. Advisably, in this case ABCG1 and ABCG4 is co-expressed at about the same level to reduce the disturbing effect of homodimer activities to the minimum. Expression levels and the amount of proteins can be set by measuring their amount using Western-blot with the selective antibodies of the invention.

The physiological substrates for ABCG1, ABCG4 and the potential heterodimer are unknown. We are currently investigating whether these transporters act individually or in complex as lipid and/or sterol transporters, as previously proposed. The Drosophila homologues (White, Scarlet and Brown) of the human ABCG1 and ABCG4 were proposed to work as heterodimers, and the White and Scarlet heterodimer are thought to transports 3-OH kynurenine from the cytoplasm into the pigment granules in the eye of the fly (Mackenzie, S. et al., 2000). Since ABCG4 is expressed in the human eye (Oldfield S et al., 2002), in the screening method described above, we tested if this compound could stimulate the ATPase activity of ABCG1, ABCG4 and ABCG1/ABCG4, co-expressed in Sf9 membranes. However, we could not detect a change in activity when the compound was added at various concentrations (data not shown).

Once inhibitors selective for any of the homodimers are used, the heterodimer activity can be measured even in the presence of “contaminating” homodimers the activities of which can be blocked by the inhibitors. Thus, the screening method of the invention can be carried out even if co-expression does not ensure that essentially only heterodimers are present.

An alternative possibility for assessing heterodimer activities will be to calculate amounts of “contaminating” homodimers present as an artifact by using selective substrates or activators of the homodimers. This method is to be applied if the tested substance seems to be a modulator (activator, inhibitor or substrate) of both the heterodimer and one of the homodimers (mentioned herein as ABCG1 or ABCG4 selective modulators). For example, if a mixture of ABCG1 homodimer and the heterodimer are present (e.g. ABCG1 is expressed in excess amount compared with ABCG4), a selective substrate of ABCG1 with a known selective activity can be used to calculate the amount of ABCG1 homodimer and thereby ABCG1/ABCG4 heterodimer in the mixture. If a further compound is tested, and if it proves not to be a transportable substrate of the ABCG1 homodimer, the ATPase activity characteristic to the heterodimer is easy to be calculated. However, even if the tested substance is a substrate of both the ABCG1 homodimer and of the heterodimer, the heterodimer transport activity for that substrate can be calculated, once homodimer activity is measured separately and once the amount of “contaminating” homodimer has been calculated by using the known selective substrate, as described above. Analogous methods can be devised using inhibitors and activators selective for one of the homodimers.

### INDUSTRIAL APPLICATION AND ADVANTAGES OF THE INVENTION

The physiological function of mammalian ABCG1 and ABCG4 are not known, yet there is a growing body of evidence that they are involved in lipid/sterol regulation (for review see Schmitz G et al., 2001). Elevated expression of ABCG1 (Klucken J et al., 2000, Venateswaran A et al., 2000, Laffitte B A et al., 2001) and ABCG4 mRNAs (Engel T et al., 2001) was identified subsequent to cholesterol loading of macrophages. Acetylated or oxidized LDL strongly induced ABCG1 expression as does their treatment with LXR and RXR agonists, oxysterols and retinoids (Engel T et al., 2001). Also, cholesterol efflux mediated by the cholesterol acceptor HDL₃ completely suppressed ABCG1 expression (Klucken J et al., 2000, Venateswaran A et al., 2000, Lorkowski S et al., 2001). Inhibition of ABCG1 expression by antisense oligonucleotides decreased HDL₃ cholesterol efflux from cells (Klucken J et al., 2000). Importantly, ABCG1 can modulate plasma lipoprotein levels in vivo. The hepatic overexpression of ABCG1 in mice, using adenovirus infection methodology, showed that ABCG1 plays an important role in cholesterol and lipoprotein metabolism by causing decreased plasma HDL levels and increased biliary cholesterol excretion (Brewer H B et al., 2003, Ito T, 2003). Direct transport experiments by using expression systems should help to clarify this important physiological role.

The role of ABCG1 and ABCG4 has been suggested in various neurological disorders, such as Alzheimer's disease and Parkinson's disease.

However, the physiological substrates for ABCG1, ABCG4 and of the ABCG1/ABCG4 heterodimer are unknown. The present invention allows screening any substances to identify whether they are modulators, i.e. activators, inhibitors or substrates of the homo or heterodimer proteins in question. For example, we are currently investigating whether these transporters act individually or in complex as lipid and/or sterol transporters, as previously proposed.

Based on the present data, future ABCG1 or ABCG4 expression studies in mammalian cells should take note of the endogenous levels of both transporters. The overexpression of one transporter or the other may dramatically influence the dimeric state of the transporters and thus influence function. We generated specific mouse polyclonal antisera that can recognize ABCG1 and ABCG4 on Western blot. Monoclonal antibodies are produced and the selectivity of the polyclonal antisera suggests that we will be able to perform studies that consider endogenous transporter levels.

Our newly developed selective antibodies can selectively recognize ABCG1 and ABCG4 protein expression. Thus, these reagents, together with the screening and detection methods of the invent will provide efficient tools to establish tissue expression, cellular localization and distribution for these proteins, as well as to identify candidate drug which may be useful in disorders related with these proteins.

### ACKNOWLEDGEMENTS

This work has been supported by research grants from OTKA and OM, Hungary (T-31952, T35926, T38337, D 45957, ETT, and NKFP). N. Barry Elkind was a recipient of a young investigator fellowship of the Hung. Acad. Sci., Balázs Sarkadi is a recipient of a Howard Hughes International Scholarship.

### REFERENCES

1. Annilo, T., Tammur, J., Hutchinson, A., Rzhetsky, A., Dean, M., and Allikmets, R. (2001) Cytogenet Cell Genet 94, 196-201
2. Bodo, A., Bakos, E., Szeri, F., Varadi, A., and Sarkadi, B. (2003) J Biol Chem 278, 23529-23537
3. Brewer, H. B., Jr., and Santamarina-Fojo, S. (2003) Am J Cardiol 91, 3E-11E
4. Chen, H., Rossier, C., Lalioti, M. D., Lynn, A., Chakravarti, A., Perrin, G., and Antonarakis, S. E. (1996) Am J Hum Genet 59, 66-75
5. Croop, J. M., Tiller, G. E., Fletcher, J. A., Lux, M. L., Raab, E., Goldenson, D., Son, D., Arciniegas, S., and Wu, R. L. (1997) ]Gene 185, 77-85
6. Dean, M., Rzhetsky A., Allikmets R. (2001) Genome Research 11, 1156-1166
7. Engel, T., Lorkowski, S., Lueken, A., Rust, S., Schluter, B., Berger, G., Cullen, P., and Assmann, G. (2001) Biochem Biophys Res Commun 288, 483-488
8. Ewart, G. D., Cannell, D., Cox, G. B., and Howells, A. J. (1994)i J Biol Chem 269, 10370-10377
9. Graf, G. A., Yu, L., Li, W. P., Gerard, R., Tuma, P. L., Cohen, J. C., and Hobbs, H. H. (2003) J Biol Chem 278, 48275-48282
10. Haimeur, A., Conseil, G., Deeley, R. G., and Cole, S. P. (2004) Curr Drug Metab 5, 21-53
11. Ilias, A., Urban, Z., Seidl, T. L., Le Saux, O., Sinko, E., Boyd, C. D., Sarkadi, B., and Varadi, A. (2002) J Biol Chem 277, 16860-16867.
12. Ito, T. (2003) Drug News Perspect 16, 490-492
13. Janvilisri, T., Venter, H., Shahi, S., Reuter, G., Balakrishnan, L., and van Veen, H. W. (2003) J Biol Chem 278, 20645-20651
14. Klein, I., Sarkadi, B., and Varadi, A. (1999) Biochim Biophys Acta 1461, 237-262
15. Klucken, J., Buchler, C., Orso, E., Kaminski, W. E., Porsch-Ozcurumez, M., Liebisch, G., Kapinsky, M., Diederich, W., Drobnik, W., Dean, M., Allikmets, R., and Schmitz, G. (2000) Proc Natl Acad Sci U S A 97, 817-822
16. Laffitte, B. A., Repa, J. I., Joseph, S. B., Wilpitz, D. C., Kast, H. R., Mangelsdorf, D. J., and Tontonoz, P. (2001) Proc Natl Acad Sci USA 98, 507-512
17. Lorkowski, S., Kratz, M., Wenner, C., Schmidt, R., Weitkamp, B., Fobker, M., Reinhardt, J., Rauterberg, J., Galinski, E. A., and Cullen, P. (2001) Biochem Biophys Res Commun 283, 821-830
18. Mackenzie, S. M., Howells, A. J., Cox, G. B., and Ewart, G. D. (2000) Genetica 108, 239-252
19. Mitomo, H., Kato, R., Ito, A., Kasamatsu, S., Ikegami, Y., Kii, I., Kudo, A., Kobatake, E., Sumino, Y., and Ishikawa, T. (2003) Biochem J 373, 767-774
20. Muller, M., Bakos, E., Welker, E., Varadi, A., Germann, U. A., Gottesman, M. M., Morse, B. S., Roninson, I. B., and Sarkadi, B. (1996) J Biol Chem 271, 1877-1883
21. Oldfield, S., Lowry, C., Ruddick, J., and Lightman, S. (2002) Biochim Biophys Acta 1591, 175-179
22. Ozvegy, C., Litman, T., Szakacs, G., Nagy, Z., Bates, S., Varadi, A., and Sarkadi, B. (2001) Biochem Biophys Res Commun 285, 111-117
23. Ozvegy, C., Varadi, A., and Sarkadi, B. (2002) J Biol Chem 277, 47980-47990
24. Sarkadi, B., Özvegy-Laczka, C., Német, K., and Váradi, A. (2004) FEBS Letters. 567(1):116-20.
25. Sarkadi, B., Price, E. M., Boucher, R. C., Germann, U. A., and Scarborough, G. A. (1992) J Biol Chem 267, 4854-4858.
26. Schmitz, G., Langmann, T., and Heimerl, S. (2001) J Lipid Res 42, 1513-1520
27. Tusnady, G. E., and Simon, I. (1998) J Mol Biol 283, 489-506
28. Venkateswaran, A., Repa, J. J., Lobaccaro, J. M., Bronson, A., Mangelsdorf, D. J., and Edwards, P. A. (2000) J Biol Chem 275, 14700-14707
29. Wang N., Lan D., Chen W., Matsuura F., Tall A R. (2004) Proc Nat'l Acad Sci 101(26), 9774-9779
30. Woehlecke, H., Pohl, A., Alder-Baerens, N., Lage, H., and Herrmann, A. (2003) Biochem J 376, 489-495
31. Yoshikawa, M., Yabuuchi, H., Kuroiwa, A., Ikegami, Y., Sai, Y., Tamai, I., Tsuji, A., Matsuda, Y., Yoshida, H., and Ishikawa, T. (2002) Gene 293, 67-75

1. A method for determining whether a substance is a selective activator, a selective inhibitor or a selective substrate of an ABCG1/ABCG4 heterodimer protein, comprising the steps of
providing an ABCG1 homodimer protein, an ABCG4 homodimer protein and the ABCG1/ABCG4 heterodimer protein in active form,
the homodimer proteins and the heterodimer protein are separately contacted with the substance under conditions appropriate for detecting activities of the proteins,
assessing activities of the homodimer proteins and of the heterodimer protein in the presence and in the absence of the substance, wherein
if, in the presence of the substance, the activity of the heterodimer protein is increased whereas the activities of the homodimer proteins are not increased or increased only to a significantly lesser extent, the substance is considered as a selective activator of the ABCG1/ABCG4 heterodimer protein,
if, in the presence of the substance, the activity of the heterodimer protein is decreased whereas the activities of the homodimer proteins are not decreased or decreased only to a significantly lesser extent, the substance is considered as a selective inhibitor of the ABCG1/ABCG4 heterodimer protein,
if the type of activity assessed is transport activity and the substance is transported by said heterodimer protein whereas it is not transported by the homodimer proteins or transported only to a significantly lesser extent, the substance is considered as a selective substrate of the ABCG1/ABCG4 heterodimer protein.

2. The method of claim 1, for determining whether a substance is a selective activator of an ABCG1 homodimer protein, an ABCG4 homodimer protein or an ABCG1/ABCG4 heterodimer protein, comprising the steps of
providing, in active form, at least two, preferably all the three dimer proteins of the following group: an ABCG1 homodimer protein, an ABCG4 homodimer protein and an ABCG1/ABCG4 heterodimer protein,
the proteins are separately contacted with the substance under conditions appropriate for detecting activities of the proteins,
assessing activities of the proteins in the presence and in the absence of the substance, wherein
if, in the presence of the substance, the activity of any one of the ABCG1 homodimer protein, the ABCG4 homodimer protein or the ABCG1/ABCG4 heterodimer is increased whereas the activities of the other two proteins are not increased or increased only to a significantly lesser extent, the substance is considered as a selective activator of the protein the activity of which is increased to the largest extent.

3. The method of claim 1, for determining whether a substance is a selective inhibitor of an ABCG1 homodimer protein, an ABCG4 homodimer protein or an ABCG1/ABCG4 heterodimer protein comprising the steps of
providing, in active form, at least two, preferably all the three dimer proteins of the following group: an ABCG1 homodimer protein, an ABCG4 homodimer protein and an ABCG1/ABCG4 heterodimer protein,
the proteins are separately contacted with the substance under conditions appropriate for detecting activities of the proteins,
assessing activities of the proteins in the presence and in the absence of the substance, wherein
if, in the presence of the substance, the activity of any one of the ABCG1 homodimer protein, the ABCG4 homodimer protein or the ABCG1/ABCG4 heterodimer is decreased whereas the activity of the other protein(s) is not decreased or decreased only to a significantly lesser extent, the substance is considered as a selective inhibitor of the protein the activity of which is decreased to the largest extent.

4. The method of claim 1, for determining whether a substance is a selective substrate of an ABCG1 homodimer protein, an ABCG4 homodimer protein or an ABCG1/ABCG4 heterodimer protein comprising the steps of
providing, in active form, at least two, preferably all the three dimer proteins of the following group: an ABCG1 homodimer protein, an ABCG4 homodimer protein and an ABCG1/ABCG4 heterodimer protein,
the proteins are separately contacted with the substance under conditions appropriate for detecting transport activities of the proteins,
assessing activities of the proteins in the presence and in the absence of the substance, wherein
if the type of activity assessed is transport activity and the substance is transported by any one of the ABCG1 homodimer protein, the ABCG4 homodimer protein or the ABCG1/ABCG4 heterodimer whereas it is neither transported by the other protein(s) or transported only to a significantly lesser extent, the substance is considered as a selective substrate of the protein having the highest transport activity.

5. The method of claim 1 wherein the type of activity assessed is ATP-ase activity.

6. The method of any of claim 1 wherein the proteins are provided in cells or cell membrane preparations wherein it is ensured that no interfering ABC transporter activities are present, or at least it is ensured that assessments are corrected for any interfering ABC transporter activities.

7. The method of claim 1, wherein the proteins are provided in insect cells or insect cell membrane preparations wherein it is ensured that no interfering ABC transporter activities are present, or at least it is ensured that assessments are corrected for any interfering ABC transporter activities.

8. The method of claim 1, wherein the substance is an anticancer agent, a receptor or channel modifier, a hormone, a neurotransmitter, a conjugate, e.g. glutathione conjugate or a conjugated bile acid, an ionophore, a peptide, a sterol, a dye, an amino acid, a peptide, a lipid, or derivative thereof.

9. (canceled)

10. (canceled)

11. (canceled)

12. (canceled)

13. (canceled)

14. A method for the preparation of an antibody selective for ABCG1 or ABCG4, wherein
an N-terminal soluble domain comprising amino acids 1-418 for ABCG1, or an at least 100 amino acid fragment thereof, is expressed as a protein fragment, or
an N-terminal soluble domain comprising amino acids 1-386 for ABCG4, or an at least 100 amino acid fragment thereof, is expressed as a protein fragment,
the expressed protein fragment is purified, and optionally pulverized and dried
the purified protein fragment is mixed with an adjuvant and injected into an animal,
if desired, the animal is boosted,
the serum obtained is recovered,
the polyclonal antibodies obtained are checked for selectivity for ABCG1 or ABCG4, respectively,
if desired, monoclonal antibodies are prepared by usual means.

15. The method of claim 14 wherein said antibody is directed to the N-terminal soluble domain, preferably to the ATP-binding domain of one of the proteins.

16. The method of claim 15 wherein said antibody is polyclonal.

17. The method of claim 15 wherein said antibody is monoclonal.

18. The method of claim 15, further comprising the steps of
contacting a biological sample with the antibody, said antibody being selective for ABCG1 or ABCG4,
detecting binding of said antibody to the ABCG1 or ABCG4 proteins, respectively, to detect ABCG1 or ABCG4 protein in the biological sample.

19. The method of claim 15, further comprising the steps of
contacting a biological sample both with an antibody selective for ABCG1 and with an antibody selective for ABCG4,
detecting binding events when both antibodies bind to the same heterodimer protein molecule,
to detect ABCG1/ABCG4 heterodimers in the biological sample.

20. A method for modulating the function or activity of an ABCG1 and/or an ABCG4 homodimer protein and/or an ABCG1/ABCG4 heterodimer protein comprising the step of substituting at least one of the subunits of said protein with
a mutant subunit of either ABCG1 or ABCG4 if the protein is an ABCG1/ABCG4 heterodimer, or
an ABCG4 subunit, or a mutant thereof, if the protein is ABCG1, or
an ABCG1 subunit, or a mutant thereof, if the protein is ABCG4,
wherein said mutant may be an inactive or an active mutant, e.g. a mutant of decreased or increased activity,
and optionally detecting an alteration in the function or activity caused by said substitution.

21. (canceled)

22. (canceled)


SEQUENCE LISTING (13 sequences)

SEQ ID NO: 1 - DNA, 24 bp, Artificial
  primer to amplify a 2038 nucleotide cDNA fragment of the long isoform of ABCG1
caccatggcctgtctgatggccgc

SEQ ID NO: 2 - DNA, 23 bp, Artificial
  primer to amplify a 2038 nucleotide cDNA fragment of the long isoform of ABCG1
tcctctctgcccggattttgtac

SEQ ID NO: 3 - DNA, 18 bp, Artificial
  PCR mutagenic primer to prepare catalytic site mutant of ABCG1
gcgtggacatgccggccc

SEQ ID NO: 4 - DNA, 19 bp, Artificial
  PCR mutagenic primer to prepare catalytic site mutant of ABCG1
gggccggcatgtccacgct

SEQ ID NO: 5 - DNA, 47 bp, Artificial
  PCR mutagenic primer to prepare catalytic site mutant of ABCG4
cgggagctgattggcatcatgggcccctcaggggctggcatgtctac

SEQ ID NO: 6 - DNA, 25 bp, Artificial
  PCR mutagenic primer to prepare catalytic site mutant of ABCG4
ggctcatcaaagaacatgacaggcg

SEQ ID NO: 7 - DNA, 29 bp, Artificial
  primer to amplify a DNA fragment encoding an intracellular region from ABCG1
atgcggatccccatggcctgtctgatggc

SEQ ID NO: 8 - DNA, 29 bp, Artificial
  primer to amplify a DNA fragment encoding an intracellular region from ABCG1
atgcctcgagtcacctcatgatgctgagg

SEQ ID NO: 9 - DNA, 25 bp, Artificial
  primer to amplify a DNA fragment encoding an intracellular region from ABCG4
atgcgaattcatggcggagaaggcg

SEQ ID NO: 10 - DNA, 34 bp, Artificial
  primer to amplify a DNA fragment encoding an intracellular region from ABCG4
atgcgcggccgctcagaggatggacaggaaggtc

SEQ ID NO: 11 - protein, 15 aa, Artificial
  short peptide sequence for immunisation against ABCG1
RKKGYKTLLKGISGK

SEQ ID NO: 12 - protein, 10 aa, Artificial
  short peptide sequence for immunisation against ABCG1
KGISGKFNSG

SEQ ID NO: 13 - protein, 10 aa, Artificial
  short peptide sequence for immunisation against ABCG4
KCLSGKFCRR